(19) United States
(12) Patent Application Publication
(10) Pub. No.: US 2017/0143861 A1
(43) Pub. Date: May 25, 2017

(54) Ammonia Reducing Agent for Commercial Aviary

(71) Applicant: Dooley Chemical, LLC, Chattanooga, TN (US)

(72) Inventors: Richard T. Jensen, Chattanooga, TN (US); David L. Halstead, Chattanooga, TN (US); Frederick E. Wilson, III, Chattanooga, TN (US)

(21) Appl. No.: 14/950,169

(22) Filed: Nov. 24, 2015

(51) Int. Cl. A61L 9/01; A61L 9/14

(52) U.S. Cl. CPC A61L 9/01; A61L 9/14; A61L 2209/21

(57) ABSTRACT

A bedding composition for treating bedding to mitigate ammonia buildup in a commercial aviary is described. The bedding composition comprises a solvent; an active component comprising: 86.5-98.9 wt % alkali metal silicate; 1-10 wt % stabilizer; 0.1-10 wt % bacteriacide; 0-2 wt % colorant; and 0-0.5 wt % de-chlorinator.

### BACKGROUND

The present invention is related to chemical compositions for reducing ammonia. More specifically, the present invention is directed to the reduction of ammonia liberated from uric acid, either directly or through the initial formation of urea, in large-scale commercial aviaries.

Commercial aviaries are well know wherein large numbers of birds, particularly chickens or turkeys, are grown in relatively confined areas for use in later operations, such as egg production, or consumption. In a typical operation litter is spread over the floor of the aviary as a bedding prior to introduction of a group of birds. As well known to those of skill in the art, the uric acid can be metabolized directly to ammonia in the presence of some bacteria or the uric acid is metabolized to urea which then decomposes to ammonia. With time the ammonia level increases thereby increasing the mortality rate of the birds and minimizing the comfort level of workers employed in the aviary. Some estimates place the optimum ammonia level at below 20 ppm in the air with levels above about 20 ppm correlating with unacceptable mortality rates.

Efforts to complex the liberated ammonia have met with some success even though this method is still not totally suitable. One such method involves the use of sodium bisulfate granules mixed in with the litter. The sodium bisulfate generates sulfuric acid in the presence of moisture and the sulfuric acid then reacts with the liberated ammonia to form ammonium sulfate thereby complexing the ammonia. Though this method can reduce the ammonia levels the unreacted sulfuric acid is very corrosive to machinery and clothing and the cost associated therewith is excessive.

There is an ongoing need for improvements in the commercial aviary business, especially, with regards to mitigating ammonia levels. Such an improvement is provided in the instant application.

### SUMMARY

It is an object of the invention to provide a composition for minimizing ammonia liberation in commercial aviaries.

It is another object of the invention to provide a composition for use with litter in a commercial aviary wherein the composition mitigates ammonia liberation from uric acid deposits.

A particular feature of the present invention is the ability to mitigate ammonia liberation from uric acid, directly or through urea, without deleterious effects on machinery, clothing or the livestock.

These and other advantages, as will be realized, are provided in a bedding composition for mitigating ammonia buildup in a commercial aviary. The bedding composition comprises a solvent; an active component comprising: 86.5-98.9 wt % alkali metal silicate; 1-10 wt % stabilizer; 0.1-10 wt % bacteriacide; 0-2 wt % colorant; and 0-0.5 wt % de-chlorinator.

Yet another embodiment is provided in a method for treating a commercial aviary comprising:

spreading litter in the commercial aviary;
applying a composition to the litter wherein the composition comprises an active component comprising alkali metal silicate; and
collecting uric acid on the litter wherein the uric acid and the composition react to litter comprising form treated uric acid.

### BRIEF DESCRIPTION OF DRAWINGS

FIG. 1 is a flow chart representation of an embodiment of the invention.

### DESCRIPTION

The present invention is directed to a composition suitable for mitigating ammonia liberation from uric acid either directly or through urea. More specifically, the present invention is related to a composition and method suitable for mitigating ammonia liberation from urea in commercial aviaries and the like.

The invention will be described with reference to the FIGURE forming an integral, non-limiting, component of the disclosure. The FIGURE is intended to facilitate an understanding of the invention and is not intended to limit the invention in any way.

The composition comprises solvent, an active component, and optional adjuvants. The solvent is chosen to be sufficient to dissolve the active components in solution with water, alcohols with 2 to 18 carbons, glycols with 2 to 18 carbons and polyethylene glycols with 4 to 25 carbons being exemplary for demonstration of the invention. Water is particularly preferred due to availability and cost.

The composition is added to litter to form a bedding for a commercial aviary. About 18 Kg of active ingredient in about 152 Kg of solvent will treat from about 28 M³ of bedding to about 141 M³ of bedding. The active ingredient is preferably applied at a level of about 0.12 Kg/M³ to about 0.6 Kg/M³ and more preferably about 0.2 Kg/M³ to about 0.31 Kg/M³ for optimum coverage.

The solvent represents 0.5 to 70 wt % of the composition prior to combining with the litter. In one embodiment the water content is kept as low as practical thereby allowing for shipment of the active components for dilution by the end user just prior to use. In this embodiment the solvent content is preferably at least 0.5 wt % to no more than 20 wt %. Below about 0.5 wt % solvent the active components may precipitate, which is undesirable, and above about 20 wt % solvent the shipping weight is unnecessarily high. It is more preferable that the composition, when prepared for shipping, comprises at least 0.5 wt % to 10 wt % and more preferably 1 wt % solvent to 3 wt % solvent. When diluted for use it is preferable that the composition comprises at least 20 wt % solvent to 50 wt % solvent and more preferably 20 wt % solvent to 50 wt % solvent with 30 wt % solvent to 40 wt % solvent being most preferred. Below about 20 wt % solvent the active components are more susceptible to clogging sprayer orifices which is undesirable. Above about 70 wt % solvent the amount of liquid deposited to achieve an adequate level of active components is excessive.

The active component represent 30 wt % to 95.5 wt % of the composition. When formulated for shipment the active component represents 80 wt % to 95.5 wt % of the composition, more preferably 90 wt % to 95.5 wt % and more preferably 97 wt % to 99 wt %. When diluted for use it is preferable that the composition comprises at least 5 wt % to 80 wt % active component, more preferably 50 wt % to 80 wt % active component and even more preferably 60 wt % to 70 wt % active component.

The active component comprises an alkali metal silicate, stabilizer, bactericide, optional colorant and option de-chlorinator. More preferably, the active component consist essentially of an alkali metal silicate, stabilizer, bactericide, optional colorant and option de-chlorinator.

The active components comprise primarily an alkali metal silicate preferably selected from the group consisting of lithium silicate, sodium silicate and potassium silicate with sodium silicate being most preferred. While not limited to any theory, the alkali metal silicate is hypothesized to encapsulate the uric acid, or urea, thereby significantly hindering the rate of ammonia liberation. The alkali metal silicate represents 86.5 to 98.9 wt % of the active component and more preferably 91.7 to 94.35 wt %. Below about 86.5 wt % the activity decreases and above 98.9 wt % the additional components of the active component are in insufficient quantity.

In addition to the alkali metal silicate the active component preferably comprises a stabilizer for the alkali metal silicate. Suitable stabilizers include alkali salts of metasilicate, alkali salts of orthophosphate, alkali salts of polyphosphates and alkali salts of hexametaphosphates. It is preferable that the active component comprises at least 1 wt % stabilizer to 10 wt %. Below about 1 wt % stabilizer the activity is insufficient and above about 10 wt % stabilizer the advantages diminish. More preferably, the active component comprises at least 5 wt % stabilizer to 7 wt % stabilizer.

It is preferable to include a bactericide in the active component. Preferred bactericides are selected from benzisothiazone derivatives, poly(hexamethylene biquanidine) derivatives, carbamate derivatives, chlorinated isophthalonitrile derivatives, chlorinated phenolic derivatives and aldehyde derivatives. A particularly suitable bactericides for demonstration of the invention is Promex 20S. It is preferable that the active component comprises at least 0.1 wt % bactericide to 10 wt %. Below about 0.1 wt % the activity is insufficient and above about 10 wt % the advantages diminish. More preferably, the active component comprises at least 0.6 wt % bactericide to 0.9 wt %.

When formulated for shipment, with low solvent content, variations in the solvent utilized for dilution must be taken into consideration. Water is a preferable solvent and it is preferable to add components to neutralize common components of water such as chlorine. It is preferable in some embodiments to add de-chlorinators such as sodium thiosulfate. The de-chlorinator is optionally present in an amount of up to about 0.5 wt %. Above about 0.5 wt % the benefits do not increase significantly. More preferably, the de-chlorinator is present in an amount of about 0.05 wt % to 0.2 wt %.

The composition is typically sprayed onto an area covered by litter or combined with litter to form bedding and the bedding is then spread over an area. As the primary components are colorless it is often difficult to insure an area is completely covered and to confirm uniformity. It is therefore desirable, in some embodiments, to include a colorant which can be a dye. The colorant is preferably non-hazardous with a light color, such as blue, or a color which his observable under special lighting, such as a UV active dye which is preferable for aesthetics. Particularly preferred colorants include Intracid violet 4BNS, Uvitex CBS-X. The colorant is optionally present in an amount of up to about 2 wt %. Above about 2 wt % the benefits do not increase significantly. More preferably, the dye is present in an amount of up to about 1 wt % and even more preferably up to about 0.1 wt %.

Adjuvants may be added to stabilize the active component or to adjust pH or rheology. Particularly preferred adjuvants include pH buffers, surfactants, markers, etc.

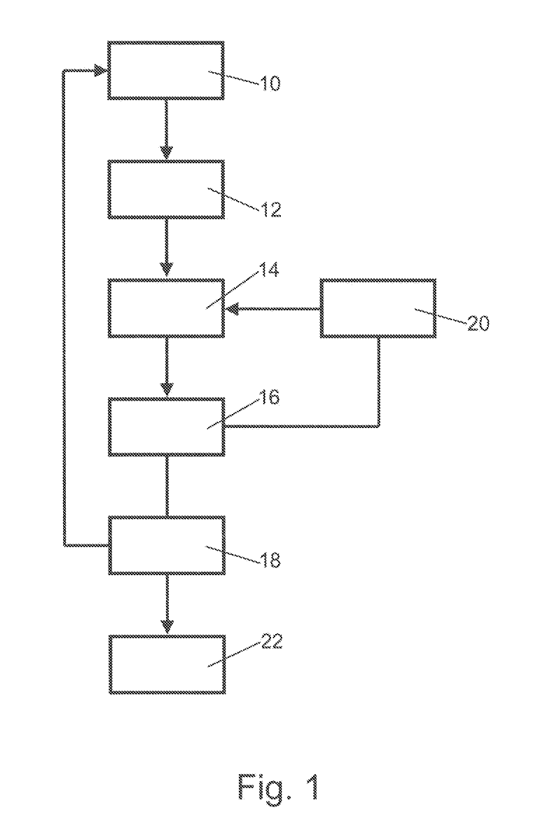

An embodiment of the invention will be described with reference to FIG. 1 wherein the embodiment is illustrated by flow chart representation. Starting with a clean commercial aviary, a layer of litter is spread over the area at 10. Litter can be selected from a large listing of commercially available materials including wood chips or shavings, sand, pellets of various materials, cellulose based materials, fibrous materials, hay, straw and the like. The inventive composition is spread over the litter at 12 preferably with uniformity. The method of spreading is not particularly limited, however, spray techniques are most preferred due to the operational simplicity and common use in such environments. The spray technique can be from a container, and preferably a pressurized container, wherein diluted active components are contained. Alternatively, concentrated active components, with minimal solvent, may be drawn from a container and integrated into a stream of flowing solvent, such as water, wherein the solvent draws the concentrate into the flowing stream of solvent such as by aspiration. It is preferable to monitor the uniformity by visible inspection of the dye incorporated in the composition as discussed elsewhere herein. Birds are introduced into the area at 14, preferably, after allowing the solvent to evaporate to a desired amount. As the birds excrete uric acid the uric acid, or urea formed from the uric acid, is believed to be stabilized by encapsulation thereby mitigating ammonia liberation. The stabilized uric acid is referred to herein as treated uric acid. After the desired maturity level of the birds is achieved the birds are removed at 16. In one embodiment the area is cleaned at 18 to remove the litter and treated urea and new litter is introduced at 10 for an additional cycle. In another embodiment, the existing litter is retreated, represented at 20, prior to introduction of another generation of birds. The litter and treated urea may be diverted and treated to form fertilizer at 22.

The invention has been described with particular reference to preferred embodiments without limit thereto. One of skill in the art would realize additional embodiments and improvements which are not specifically enumerated but which are within the scope of the invention as specifically set forth in the claims appended hereto.

1. A bedding composition for mitigating ammonia buildup in a commercial aviary comprising:
a solvent;
an active component comprising:
86.5-98.9 wt % alkali metal silicate;
1-10 wt % stabilizer;
0.1-10 wt % bactericide;
0-2 wt % colorant; and
0-0.5 wt % de-chlorinator.

2. The bedding composition for mitigating ammonia buildup in a commercial aviary of claim 1 further comprising litter.

3. The bedding composition for mitigating ammonia buildup in a commercial aviary of claim 2 comprising 0.12 Kg of said active component per M³ of litter to about 0.6 Kg of said active component per M³.

4. The bedding composition for mitigating ammonia buildup in a commercial aviary of claim 3 comprising 0.2 Kg of said active component per M³ of litter to about 0.31 Kg of said active component per M³ of litter.

5. The bedding composition for mitigating ammonia buildup in a commercial aviary of claim 1 comprising 0.5 to 70 wt % said solvent.

6. The bedding composition for mitigating ammonia buildup in a commercial aviary of claim 5 comprising:
0.5 wt %-20 wt % solvent.

7. The bedding composition for mitigating ammonia buildup in a commercial aviary of claim 6 comprising 0.5 wt %-10 wt % solvent.

8. The bedding composition for mitigating ammonia buildup in a commercial aviary of claim 7 comprising 1 wt %-3 wt % solvent.

9. The bedding composition for mitigating ammonia buildup in a commercial aviary of claim 5 comprising 20 wt %-70 wt % solvent.

10. The bedding composition for mitigating ammonia buildup in a commercial aviary of claim 9 comprising 20 wt %-50 wt % solvent.

11. The bedding composition for mitigating ammonia buildup in a commercial aviary of claim 10 comprising 30 wt %-40 wt % solvent.

12. The bedding composition for mitigating ammonia buildup in a commercial aviary of claim 1 wherein said solvent is water.

13. The bedding composition for mitigating ammonia buildup in a commercial aviary of claim 1 comprising 30 wt %-95.5 wt % said active component.

14. The bedding composition for mitigating ammonia buildup in a commercial aviary of claim 13 comprising 80 wt %-95.5 wt % said active component.

15. The bedding composition for mitigating ammonia buildup in a commercial aviary of claim 14 comprising 90 wt %-95.5 wt % said active component.

16. The bedding composition for mitigating ammonia buildup in a commercial aviary of claim 15 comprising 97 wt %-95.5 wt % said active component.

17. The bedding composition for mitigating ammonia buildup in a commercial aviary of claim 1 wherein said active component comprises 91.7 wt %-94.35 wt % said alkali metal silicate.

18. The bedding composition for mitigating ammonia buildup in a commercial aviary of claim 1 wherein said alkali metal silicate is selected from the group consisting of lithium silicate, sodium silicate and potassium silicate.

19. The bedding composition for mitigating ammonia buildup in a commercial aviary of claim 18 wherein said alkali metal silicate is sodium silicate.

20. The bedding composition for mitigating ammonia buildup in a commercial aviary of claim 1 wherein said active component comprises 5 wt %-7 wt % said stabilizer.

21. The bedding composition for mitigating ammonia buildup in a commercial aviary of claim 1 wherein said stabilizer is selected from the group consisting of alkali salts of metasilicate, alkali salts of orthophosphate, alkali salts of polyphosphate and alkali salts of hexametaphosphates.

22. The bedding composition for mitigating ammonia buildup in a commercial aviary of claim 1 wherein said active component comprises 0.6 wt %-0.9 wt % said bacteriacide.

23. The bedding composition for mitigating ammonia buildup in a commercial aviary of claim 1 wherein said bacteriacide is selected from benzisothiazone derivatives, poly(hexamethylene biquanidine) derivatives, carbamate derivatives, chlorinated isophthalonitrile derivatives, chlorinated phenolic derivatives and aldehyde derivatives.

24. The bedding composition for mitigating ammonia buildup in a commercial aviary of claim 1 wherein said active component comprises 0 wt %-1 wt % said colorant.

25. The bedding composition for mitigating ammonia buildup in a commercial aviary of claim 24 wherein said active component comprises 0 wt %-0.2 wt % said colorant.

26. The bedding composition for mitigating ammonia buildup in a commercial aviary of claim 1 wherein said colorant is selected from Intracid violet 4BNS and Uvitex CBS-X.

27. The bedding composition for mitigating ammonia buildup in a commercial aviary of claim 1 wherein said active component comprises 0.05 wt %-0.2 wt % said de-chlorinator.

28. The bedding composition for mitigating ammonia buildup in a commercial aviary of claim 1 wherein said de-chlorinator is sodium thiosulfate.

29. The bedding composition for mitigating ammonia buildup in a commercial aviary of claim 1 further comprising an adjuvant.

30. A method for treating a commercial aviary comprising:
spreading litter in said commercial aviary;
applying a composition to said litter wherein said composition comprises an active component comprising alkali metal silicate; and
collecting uric acid on said litter wherein said uric acid and said composition react to litter comprising form treated uric acid.

31. The method for treating a commercial aviary of claim 30 wherein said applying said composition is after said spreading of said litter.

32. The method for treating a commercial aviary of claim 30 further comprising reapplying said composition to said litter comprising said treated uric acid.

33. The method for treating a commercial aviary of claim 30 wherein said applying said composition comprises spraying.

34. The method for treating a commercial aviary of claim 33 wherein said spraying comprises spraying a diluted active component.

35. The method for treating a commercial aviary of claim 33 wherein said spraying comprises spraying a concentrated active component.

36. The method for treating a commercial aviary of claim 30 comprising 0.12 Kg of said active component per M³ of litter to about 0.6 Kg of said active component per M³.

37. The method for treating a commercial aviary of claim 36 comprising 0.2 Kg of said active component per M³ of litter to about 0.31 Kg of said active component per M³.

38. The method for treating a commercial aviary of claim 30 wherein said composition further comprises a solvent.

39. The method for treating a commercial aviary of claim 38 comprising 0.5 to 70 wt % said solvent.

40. The method for treating a commercial aviary of claim 38 comprising: 0.5 wt %-20 wt % solvent.

41. The method for treating a commercial aviary of claim 40 comprising 0.5 wt %-10 wt % solvent.

42. The method for treating a commercial aviary of claim 41 comprising 1 wt %-3 wt % solvent.

43. The method for treating a commercial aviary of claim 38 comprising 20 wt %-70 wt % solvent.

44. The method for treating a commercial aviary of claim 43 comprising 20 wt %-50 wt % solvent.

45. The method for treating a commercial aviary of claim 44 comprising 30 wt %-40 wt % solvent.

46. The method for treating a commercial aviary of claim 38 wherein said solvent is water.

47. The method for treating a commercial aviary of claim 30 wherein said composition comprises 30 wt %-95.5 wt % said active component.

48. The method for treating a commercial aviary of claim 30 wherein said composition comprises 80 wt %-95.5 wt % said active component.

49. The method for treating a commercial aviary of claim 48 wherein said composition comprises 90 wt %-95.5 wt % said active component.

50. The method for treating a commercial aviary of claim 49 wherein said composition comprises 97 wt %-95.5 wt % said active component.

51. The method for treating a commercial aviary of claim 30 wherein said active component comprises 86.5-98.9 wt % said alkali metal silicate.

52. The method for treating a commercial aviary of claim 51 wherein said active component comprises 91.7 wt %-94.35 wt % said alkali metal silicate.

53. The method for treating a commercial aviary of claim 30 wherein said alkali metal silicate is selected from the group consisting of lithium silicate, sodium silicate and potassium silicate.

54. The method for treating a commercial aviary of claim 53 wherein said alkali metal silicate is sodium silicate.

55. The method for treating a commercial aviary of claim 30 wherein said active component comprises 1-10 wt % stabilizer.

56. The method for treating a commercial aviary of claim 55 wherein said active component comprises 5 wt %-7 wt % said stabilizer.

57. The method for treating a commercial aviary of claim 55 wherein said stabilizer is selected from the group consisting of alkali salts of metasilicate, alkali salts of orthophosphate, alkali salts of polyphosphate and alkali salts of hexametaphosphates.

58. The method for treating a commercial aviary of claim 30 wherein said active component comprises 1-10 wt % bactericide.

59. The method for treating a commercial aviary of claim 58 wherein said active component comprises 0.6 wt %-0.9 wt % said bactericide.

60. The method for treating a commercial aviary of claim 58 wherein said bacteriacide is selected from benzisothiazone derivatives, poly(hexamethylene biquanidine) derivatives, carbamate derivatives, chlorinated isophthalonitrile derivatives, chlorinated phenolic derivatives and aldehyde derivatives.

61. The method for treating a commercial aviary of claim 30 wherein said active component comprises 0-2 wt % colorant.

62. The method for treating a commercial aviary of claim 61 wherein said active component comprises 0 wt %-1 wt % said colorant.

63. The method for treating a commercial aviary of claim 30 wherein said active component comprises 0 wt %-0.2 wt % said colorant.

64. The method for treating a commercial aviary of claim 63 wherein said colorant is selected from Intracid violet 4BNS and Uvitex CBS-X.

65. The method for treating a commercial aviary of claim 30 wherein said active component comprises 0-0.5 wt % de-chlorinator;

66. The method for treating a commercial aviary of claim 65 wherein said active component comprises 0.05 wt %-0.2 wt % said de-chlorinator.

67. The method for treating a commercial aviary of claim 65 wherein said de-chlorinator is sodium thiosulfate.

68. The method for treating a commercial aviary of claim 30 wherein said composition further comprises adjuvant.
